(19) United States
(12) Patent Application Publication
(10) Pub. No.: US 2025/0032606 A1
(43) Pub. Date: Jan. 30, 2025

(54) POLYVALENT VACCINES AND METHODS FOR MAKING THEM

(71) Applicants: David S. CAMPO, Atlanta, GA (US); Richard URBANOWICZ, Liverpool, Lancashire (GB); Mounir ABOUHAIDAR, Toronto (CA); UNIVERSITY HEALTH NETWORK, Toronto (CA); THE GOVERNING COUNCIL OF THE UNIVERSITY OF TORONTO, Toronto (CA); UNIVERSITY OF LIVERPOOL, Liverpool, Lancashire (GB); CENTERS FOR DISEASE CONTROL AND PREVENTION, Atlanta, GA (US); THE UNITED STATES OF AMERICA, AS REPRESENTED BY THE SECRETARY, DEPARTMENT OF HEALTH AND HUMAN SERVIC, Bethesda, MD (US)

(72) Inventors: David S. CAMPO, Atlanta, GA (US); Richard A. URBANOWICZ, Liverpool, Lancashire (GB); Mounir G. ABOUHAIDAR, Toronto (CA); Alexander I. MOSA, Toronto (CA); Jordan J. FELD, Toronto (CA)

(21) Appl. No.: 18/709,405

(22) PCT Filed: Nov. 14, 2022
(86) PCT No.: PCT/CA2022/051680

§ 371 Date: May 10, 2024

Related U.S. Application Data
(60) Provisional application No. 63/278,467, filed on Nov. 11, 2021.

(51) Int. Cl. A61K 39/29; A61K 39/00; A61K 39/385; A61P 31/14; C07K 14/005; C12N 7/00; G16B 15/30; G16B 40/30

(52) U.S. Cl. CPC A61K 39/29; A61K 39/385; A61P 31/14; C07K 14/005; C12N 7/00; G16B 15/30; G16B 40/30; A61K 2039/6081; A61K 2039/70; C12N 2770/24222; C12N 2770/24234

(57) ABSTRACT

There is described herein polyvalent HCV vaccines, preferably comprising SEQ ID Nos. 1-5. There is also described herein methods of designing polyvalent vaccines.

### FIELD OF THE INVENTION

The invention relates to polyvalent vaccines and methods of making them, including specific polyvalent vaccines against hepatitis C virus (HCV).

### BACKGROUND OF THE INVENTION

Hepatitis C is a leading cause of morbidity and mortality from liver disease worldwide (1). The introduction of curative, direct-acting antivirals spurred hopes for global HCV elimination (2). However, with an estimated 1.75 million new infections and 400,000 deaths annually, it may be challenging to achieve the World Health Organization's 2030 elimination targets with treatment alone (3). Availability of an effective HCV vaccine would significantly aid in these efforts (4).

Vaccine development has been impeded, however, by the extreme genetic variability of HCV, which renders immune responses produced against one variant ineffective against others (5, 6). Though classified at the full genomic level into eight genotypes differing at 30-35% of nucleotide positions, HCV's heterogeneity is not distributed uniformly along the genome (7). The most heterogeneous region, Hypervariable Region 1 (HVR1), encodes the N-terminal 27 amino acid (aa) portion of the envelope protein E2 (8). Though HVR1 contains an immunodominant neutralizing epitope, mediates interactions with the HCV co-receptor Scavenger Receptor class B type 1 (SRB1), and is strongly positively selected in natural infection, its application to vaccine development has been limited due its extraordinary genetic variability (9, 10, 11, 12). Thus, despite the capacity of anti-HVR1 antibodies to prevent homologous infection, and the favourable accessibility of this epitope to neutralizing antibodies, vaccine efforts have been focused on eliciting antibodies to conserved regions outside of HVR1 (13, 14). However, even conserved regions seem to be affected by HVR1, which physically shields conserved neutralizing epitopes, modulates envelope conformation, and elicits strain-specific, dominant “decoy” immune responses, thus suppressing recognition of the conserved subdominant epitopes (15, 16, 17). Simply removing HVR1 from E2 did not improve responses following vaccination, but instead was inferior to native E2 in terms of neutralization, possibly related to conformational changes in E2 caused by the HVR1 excision or by disruption of discontinuous antigenic epitopes involving HVR1 (17, 18, 19).

The role of HVR1 in HCV neutralization, both as a dominant epitope and as a modifier of the response to conserved epitopes, must therefore be considered in the design of any HCV vaccine.

### SUMMARY OF THE INVENTION

A hepatitis C virus (HCV) vaccine is urgently needed. Vaccine development has been hindered by HCV's genetic diversity, particularly within the immunodominant hypervariable region 1 (HVR1). Here, we developed a new strategy to elicit broadly neutralizing antibodies to HVR1, which had previously been considered infeasible.

There is described herein a novel strategy to overcome the challenge of virus heterogeneity. Using a novel information theory-based distance we modelled HVR1 genetic variability and observed discrete, genotype-independent clusters. We selected 5 central sequences from these clusters to synthesize peptides for vaccination. The mixture of HVR1 variants resulted in an antibody response that was more broadly neutralizing than each individual variant or pooled sera, indicating a synergistic interaction among immune responses to related, but distinct, HVR1 variants. These findings open a new path for the development of an HCV vaccine using sequence complementary variants of genetically divergent HVR1 antigenic epitopes.

In an aspect, there is provided a peptide comprising the sequence set forth in any one of SEQ ID Nos. 1-5 (FIG. 2B).

In a further aspect, there is provided a nucleic acid encoding for the peptides described herein and vectors comprising said nucleic acid.

In an aspect, there is provided a vaccine composition comprising one, some or all of the peptides and/or nucleic acids described herein, along with a pharmaceutically acceptable carrier and/or adjuvant.

In an aspect, there is provided the vaccine composition described herein, for use in the immunization of a subject against HCV infection.

In an aspect, there is provided a use of the vaccine composition described herein, in the preparation of a medicament for the immunization of a subject against HCV infection.

In an aspect, there is provided a method of immunizing a subject against HCV infection comprising administrating to the subject, the vaccine composition described herein.

In an aspect, there is provided a method for producing a multivalent vaccine comprising a plurality of peptides, or the nucleic acids encoding them, the method comprising: selecting a target epitope; mapping a sequence space for the targeted epitope; synthesizing peptides covering the sequence space; immunizing animals with the peptides; evaluating cross-reactivity between animal sera to determine a predictive feature of reactivity; creating a network of haplotypes wherein distance between nodes is based on the predictive feature; creating clusters of haplotypes using a mathematical model; selecting a representative haplotype from each cluster for the plurality of peptides, or the nucleic acids encoding them, in the multivalent vaccine.

In an aspect, there is provided a multivalent HCV vaccine composition produced by the method described herein.

### BRIEF DESCRIPTION OF FIGURES

These and other features of the preferred embodiments of the invention will become more apparent in the following detailed description in which reference is made to the appended drawings wherein:

FIG. 1 shows association of Genetic Distance and Cross-Reactivity. A) Overview of the cross-reactivity experiment (For more details see Campo et al (20)), which generated a total of 26,833 HVR1 pairwise cross-immunoreactive assays. B). Ratio between mean distance of non-cross reactive pairs and cross-reactive pairs using different types of distances: Hamming, BLOSUM62 scores, MIH and Euclidean distance of 5 physicochemical factors (F1, polarity; F2, secondary structure; F3, molecular size; F4, codon diversity; F5, charge) (21). C). HVR1 Information matrix using the entire sequence dataset. The diagonal shows the Shannon entropy of each position and the other entries of the matrix show the Mutual information among all pairs of positions.

FIG. 2 shows A) K-step network of global HVR1 sequence space. All non-redundant HVR1 sequences (12,245) pooled across datasets were used to construct a k-step network with nodes colored by stage of infection and scaled by haplotype frequency. B) SEQ ID Nos. 1-5 from genotype-independent clusters, selected to synthesize peptides.

FIG. 3 shows clusters in the HVR1 sequence space. A) Histogram of distances among all pairs of sequences. Three types of distances are considered: Hamming, MIH and Euclidean distances between physiochemical profiles. Each distance type is normalized by dividing by its maximum value. B) Scatterplot of the goodness of each clustering (gap Z score) according to the number of clusters. C) k-step network of all HVR1 sequences. Nodes are colored by membership to each cluster and the big nodes correspond to the most central one in each cluster.

FIG. 4 shows self and cross-reactivity of HVR1 Antigens. Mice were immunized with monovalent or pentavalent immunogens conjugated to KLH and formulated with either complete (CFA) or incomplete (IFA) Freunds adjuvant and terminally bled at day 48 (A) to evaluate anti-immunogen (HVR1-KLH) titers (B). Sera from each group were evaluated for self and cross-reactivity to each of the five antigens used for immunizations (M1-5) and patient derived control (L47), with homologous monovalent sera shown in red, and pentavalent in blue (C). Pentavalent sera were incubated with peptides containing either the immunogen (FL+KLH), full-length HVR1 alone (FL), or the c-terminal eight AA of HVR1 (C8) to measure binding inhibition to immunogen-coated ELISA plates (D). Error bars indicate mean with standard deviation. * P<0.05.

FIG. 5 shows pentavalent Sera Broadly Cross-React with Antigenically Diverse Panel of HVR1 Peptides. HCV variants with the greatest pairwise divergence in their eight C-terminal aa from each peptide used in the pentavalent formulation (A) were synthesized and used to evaluate pentavalent cross-immunoreactivity (blue circles) compared to adjuvant control (black diamonds) (B). Error bars indicate the mean with standard deviation. Dotted line indicates two times the SD of adjuvant control. *, P<0.05.

FIG. 6 shows HCVpp neutralization sensitivity, ranked from most neutralization sensitive (Tier 1) to least neutralization sensitive (Tier 4). Blue highlighting denotes the HCVpp selected for neutralization assays.

FIG. 7 shows pentavalent Sera Neutralize Panel of Antigenically Diverse HCVpp in Excess of gpE2 Vaccine. Neutralizing activity of pentavalent sera against a multi-genotype panel of HCVpp was evaluated in serial dilutions starting at 1:50, with the exception of 4.1.1 which was additionally tested at 1:20 (A). Neutralizing potencies (ID50s) were compared between pentavalent sera and sera obtained from mice immunized with a gpE2 vaccine candidate (B). The ID50 of pentavalent sera was evaluated as a function of the minimal Hamming distance between each HCVpp HVR1 (C-terminal eight aa) and the pentavalent peptides (C). Error bars indicate standard deviation. *, P<0.05.

FIG. 8 shows pentavalent sera neutralize variants resistant to neutralization by its monovalent constituents. Neutralizing potencies (ID50s) were compared across monovalent (orange) and pentavalent (blue) groups (A). Neutralizing potencies (ID50s) were compared between pentavalent sera and sera obtained from mice immunized with the same immunogens sequentially (B). Error bars indicate mean with standard deviation. *, P<0.05.

FIG. 9 shows a flow chart of the method for designing a polyvalent vaccine.

FIGS. 10A and 10B show two models of polyvalent vaccine immune response.

### DETAILED DESCRIPTION

In the following description, numerous specific details are set forth to provide a thorough understanding of the invention. However, it is understood that the invention may be practiced without these specific details.

Briefly, on the hypothesis that physicochemical rather than sequence constraints within hypervariable epitopes could be targeted by multivalent vaccines we sought to develop a novel approach to immunogen selection for multivalent vaccines. A physicochemical attribute such as polarity, charge, hydrophobicity etc may be required for function but also may be generated with a wide variety of sequences. Accordingly, we generated physicochemically distinct, epitope-matched HVR1 peptide immunogens maximally differing, within our sequence library, in the physicochemical trait of average non-bonded free energy (POLARF1). Our objective was to formulate these peptides into a multivalent vaccine capable of eliciting broadly neutralizing antibodies (nAB) by targeting “epitope-cluster”-specific residues, as well as physicochemical signatures conserved across all HVR1 “epitope-clusters”.

Following mouse immunizations, we observed cross-genotypic neutralization against HCV variants differing from the immunogen sequences by more than 70% at the amino acid level. Further, neutralization breadth and potency appeared greater for the multivalent formulation than either monovalent constituent individually, or pooled.

Based on these findings we sought to develop a more theoretically robust approach to immunogen selection based on global HVR1 cross-reactivity data.

Applicant describes herein that the global HVR1 sequence space can be modelled such that haplotype distances reflect immunological differences between HVR1 variants. We identified the parameter that best predicts cross-reactivity between two haplotypes, Mahalanobis hamming distance (MIH), and generated a network of the global sequence space using that parameter.

Applicant shows that vaccination with immunogens maximizing coverage of this space will expand neutralizing Ab breadth by favouring affinity maturation of clonal-lines with broad reactivity against haplotypes within a given cross-reactive cluster, and therefore greater overall antigenic coverage than would be generated by generating B-cell populations reactive to specific, conserved epitopes. We generated a polyvalent vaccine maximizing coverage of the network. We further evaluated if neutralization breadth induced by a polyvalent candidate exceeds its monovalent constituents, or a promising gpE2 vaccine expressed in mammalian cells.

Particularly, Applicant first applied a novel information theory-based measure of genetic distance to evaluate phenotypic relatedness between HVR1 variants. These distances were used to model HVR1's sequence space, which was found to be pentamodular, suggesting the existence of five major structural shapes. Variants from each shape were combined to pentavalently immunize mice. Sera obtained following immunization neutralized every variant in a diverse HCVpp panel (n=10), including those resistant to monovalent immunization, and at higher mean titers (ID₅₀=435) than a promising glycoprotein E2 (ID₅₀=205) vaccine. This synergistic immune response offers a novel approach to overcoming antigenic variability, and may be applicable to other highly mutable viruses

In an aspect, there is provided a peptide comprising the sequence set forth in any one of SEQ ID Nos. 1-5.

It will be understood by the skilled person that some substitutions, insertions or deletions in SEQ ID Nos. 1-5 are possible without affecting their function. Accordingly, the present invention includes peptides that comprise sequences that share at least 80%, 85%, 90%, 95%, 98%, and 99% sequence identity to SEQ ID Nos. 1-5.

In some embodiments, the peptide consists of the sequence set forth in any one of SEQ ID Nos. 1-5.

In some embodiments, the peptide is conjugated to a vaccine-suitable carrier protein. In some embodiments, the carrier protein is N-terminally conjugated. In other embodiments, the carrier protein is C-terminally conjugated.

In some embodiments, the carrier protein is keyhole limpet hemocyanin (KLH).

In some embodiments, the peptide is conjugated to KLH via a suitable linker, preferably a maleimide linkage.

In another aspect, there is provided a nucleic acid encoding the peptides described herein, as well as vectors comprising said nucleic acids. Vaccines comprising these nucleic acids could be administered as multiple mRNA, or as a single mRNA encoding cleavage signals for host signal peptidase individuation into multiple peptides. They could also be administered as DNA using approaches known in the art (either multiple different viral vectors delivering the DNA, or a single vector encoding all 5. They could also be delivered as mRNA in complex with other proteins, that may serve as adjuvants or as a structural scaffold.

In an aspect, there is provided a vaccine composition comprising at least one of the peptides described herein, along with a pharmaceutically acceptable carrier.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

In some embodiments, the vaccine comprises peptides comprising all of SEQ ID Nos. 1-5. The vaccine composition may comprise two or more, three or more, or four or more of the peptides described herein. In some embodiments, the vaccine composition comprises a peptide comprising the sequence set forth in SEQ ID No. 1, a peptide comprising the sequence set forth in SEQ ID No. 2, a peptide comprising the sequence set forth in SEQ ID No. 3, a peptide comprising the sequence set forth in SEQ ID No. 4 and a peptide comprising the sequence set forth in SEQ ID No. 5. Alternatively, the vaccine may comprise the corresponding nucleic acids encoding any of the foregoing one or more peptides.

The vaccine composition may comprise at least two different peptides, wherein the at least two different peptides comprise two of SEQ ID Nos. 1-5. The vaccine composition may comprise at least three different peptides wherein the at least three different peptides comprise three of SEQ ID Nos. 1-5. The vaccine composition may comprise at least four different peptides wherein the at least four different peptides comprise four of SEQ ID Nos. 1-5. The vaccine composition may comprise at least five different peptides wherein the at least five different peptides comprise all five of SEQ ID Nos. 1-5. Alternatively, the vaccine may comprise the corresponding nucleic acids encoding any of the foregoing one or more peptides.

The vaccine composition may include an adjuvant.

The terms “adjuvant” and “immune stimulant” are used interchangeably herein, and are defined as one or more substances that cause stimulation of the immune system. In this context, an adjuvant is used to enhance an immune response to one or more vaccine antigens/isolates. Accordingly, “adjuvants” are agents that nonspecifically increase an immune response to a particular antigen, thus reducing the quantity of antigen necessary in any given vaccine, and/or the frequency of injection necessary in order to generate an adequate immune response to the antigen of interest. In this context, an adjuvant is used to enhance an immune response to one or more vaccine antigens/isolates.

In an aspect, there is provided the vaccine composition described herein, for use in the immunization of a subject against HCV infection.

In an aspect, there is provided a use of the vaccine composition described herein, in the preparation of a medicament for the immunization of a subject against HCV infection.

In an aspect, there is provided a method of immunizing a subject against HCV infection comprising administrating to the subject, the vaccine composition described herein.

In an aspect, there is provided a method for producing a multivalent vaccine comprising a plurality of peptides, or the nucleic acids encoding them, the method comprising: selecting a target epitope; mapping a sequence space for the targeted epitope; synthesizing peptides covering the sequence space; immunizing animals with the peptides; evaluating cross-reactivity between animal sera to determine a predictive feature of reactivity; creating a network of hapolotypes wherein distance between nodes is based on the predictive feature; creating clusters of hapolotypes using a mathematical model; selecting a representative hapolotype from each cluster for the plurality of peptides, or the nucleic acids encoding them, in the multivalent vaccine.

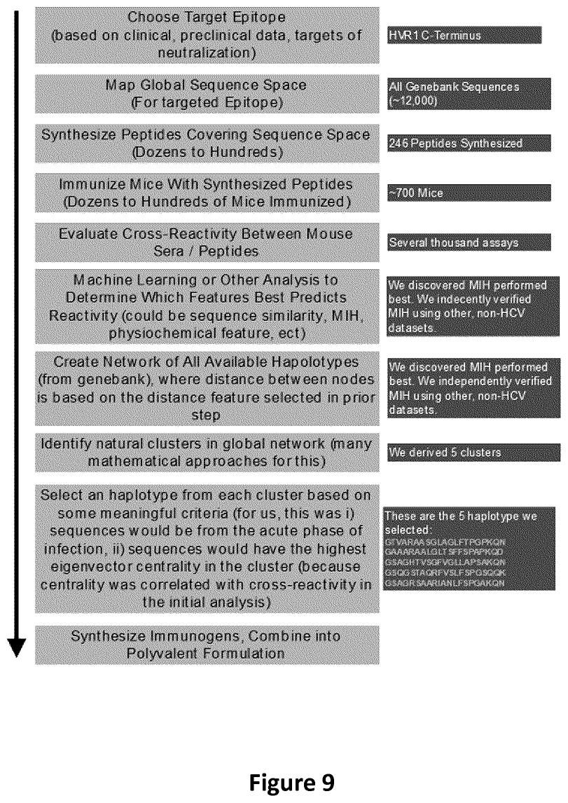

FIG. 9 shows a flowchart summarizing a specific embodiment of the design method for polyvalent vaccines.

In some embodiments, the predictive feature is sequence similarity, physicochemical, or Mahalanobis Hamming Distance (MIH). Preferably, the predictive feature is Mahalanobis Hamming Distance (MIH).

In some embodiments, clusters of hapolotypes are created using the Girvan-Newman algorithm, minimum-cut method, hierarchical clustering, modularity maximization or clique-based method.

In some embodiments, a representative haplotype from each cluster is selected based on the variant from the acute-phase of infection, and/or the sequence with the highest eigenvector centrality in the cluster.

In some embodiments, the method further comprises synthesizing the plurality of peptides or the nucleic acids encoding them.

In some embodiments, the method further comprises formulating the plurality of peptides, or the nucleic acids encoding them, into a vaccine composition.

In some embodiments, the target epitope is from HCV.

In an aspect, there is provided a multivalent HCV vaccine composition produced by the method described herein.

The advantages of the present invention are further illustrated by the following examples. The examples and their particular details set forth herein are presented for illustration only and should not be construed as a limitation on the claims of the present invention.

### Examples

### Materials and Methods

### HVR1 Sequences

All the HVR1 nucleotide sequences covering the Hypervariable region (81 bp) were obtained from the Virus Pathogen Database and Analysis Resource (ViPR) (40). In addition, the following sequences were added from previous studies: 119 sequences obtained from patients with recent HCV infection, 256 sequences from chronic HCV infection, and 262 sequences from our previously published cross-reactivity experiment (20, 41).

This set of 12,245 sequences belongs to all known HCV genotypes. All sequences were translated and cleaned in the following manner: (i) only one sequence per patient was allowed, (ii) only sequences without insertions or deletions were allowed, (iii) sequences with Ns or non-coding regions were removed. Finally, there were 969 distinct variants of the C-terminal HVR1 portion including eight amino acid sites. These variants were used in all analyses conducted here.

### Distance Between HVR1 Variants

Genetic distances based on physical-chemical properties (21) were calculated as described in (22). The MIH distance between every pair of variants was recently developed (23). The MIH is a distance based on the Mahalanobis distance that can be applied to any type of categorical data like nucleotide or amino acid sequences. The Mahalanobis distance accounts for the fact that the variance of each variable is different and that there may be covariance between variables. This distance is reduced to the Euclidean distance for uncorrelated variables with unit variance.

The MIH distance considers the variability of each position as measured by entropy and the existence of coordinated substitutions as measured by mutual information. The MIH distance between two sequences x and y is given by the following formula:

$$MIH ( x , y ) = {xy}^{T} \cdot {InfMat}^{- 1} \cdot xy$$

Where xy is the mismatch vector (with 1 where the symbols are different and 0 where they are the same) and xy^{T} is its transposed form; InfMat is the information matrix, with entropy in the diagonals and mutual information between position pairs in all other entries. Effectively, if the difference between two sequences occurs at a variable position, this difference receives a low weight. In the same manner, if the difference occurs at positions that are highly associated, this difference also receives a low weight. Thus, the MIH distance is reduced to the Hamming distance when the positions have maximum entropy, and every pair of positions has mutual information equal to zero. The MIH distance showed the best performance separating known grouping in a biological validation dataset (23).

### K-Step Network and Clustering

For the set of HVR1 variants we visualized the matrix of MIH distances by means of a k-step network as previously described (42-44). The k-step network is equivalent to the union of all possible Minimum Spanning Trees and allows for efficient visualization of the distances among all variants present in a sample. This network was then split into clusters using the Girvan-Newman method as implemented in GEPHI, which was also used to draw the networks (45). The number of clusters was chosen by using the gap statistic: for each desired number of clusters (from 2 to 40), we measured the average distance within clusters in the k-step network and compared it with the distance in 10000 random partitions of the same size (46).

### Immunizations

Peptides for immunization experiments were synthesized using Fmoc chemistry, conjugated to keyhole limpet hemocyanin (KLH) via maleimide linkage, and combined in a 1:1 emulsion with Freund's complete (primary) or incomplete (booster) adjuvant as previously described (47). For immunizations, female Balb/c mice (4-6 week years old) were ordered through the UHN animal care facility, acclimatized for one week, pre-bled, then subcutaneously injected (25 μg peptide+25 μL adjuvant) at days 0, 28, and 38, with terminal bleed via cardiac puncture at day 48 [3 mice per group-protocol approved by University Health Network (UHN) Animal Care Committee (ACC)]. Mock immunizations were performed with adjuvant and sterile PBS. Both pre-bleed and mock-immunized sera served as controls in subsequent assays. To obtain sera in all groups, blood samples were processed by centrifugation, heat-inactivated, and stored at −80° C. until analysis was performed.

### ELISA Assessment of HVR1 Binding

As previously described, ELISA was performed to measure HVR1-specific antibody responses in mouse sera (48). Briefly, 96-well plates (MaxiSorp, Thermo Fisher Scientific), were coated overnight with 2 μg/mL of HVR1 peptides at 4° C. The next morning, plates were washed 5× with PBS containing 0.05% Tween 20 (PBST) and incubated with group-pooled, serially diluted mouse (PBST) sera for 1 hour at room temperature. Post-incubation, plates were washed 5× with PBST, and incubated for 1 hour with a 1:10,000 dilution of HRP-conjugated anti-mouse IgG secondary antibody. After a final 5 washes, 3,3′,5,5′-tetramethylbenzidine (TMB) substrate was added to each well, dark-incubated for 15 min, then the reaction was terminated with Stop-Solution (0.16 M sulfuric acid). Absorbance was read at 450 nm, in triplicate, with measurements corresponding to visual colour change in each well. For competitive ELISA, the same protocol was followed, except for the additional incubation of inhibiting peptides (C-terminal 8 AA of HVR1, full-length (FL) HVR1, or FL-HVR1 conjugated to KLH) with diluted sera for 1 hour prior to plate application. ELISA cut-off was calculated by multiplying (2×) the mean of negative controls (adjuvant immunized sera). Statistical analysis was done by unpaired t-test using Prism8 software (49).

### Neutralization Assays

HCVpp neutralization assays were performed as previously described (26). Briefly, HCVpp were generated by co-transfecting HEK 293T cells with the pNL4-3.lucR⁻E⁻ packaging plasmid and expression plasmids encoding patient-derived E1E2. To test sera for neutralizing activity, Huh7 cells were plated in 96-well plates (15,000 per well), and incubated overnight. The following day, HCVpp were incubated with heat-inactivated, group-pooled, serially diluted mouse serum for 1 hour at 37° C., and then added in triplicate to Huh7 plated wells. Plates were then incubated in a CO₂ incubator at 37° C. for 4 hours before media was replaced. 72 hours later, media was removed and cells were lysed using cell lysis buffer (Promega, Southampton, UK) and placed on a rocker for 15 min. Luciferase activity was then measured in relative light units (RLUs) using a FLUOstar Omega plate reader (BMG Labtech, Aylesbury, UK) with MARS software. Each sample was tested in triplicate. The ID50 was calculated as the serum dilution that caused a 50% reduction in relative light units compared to pseudoparticles incubated with pre-bleed serum. Values were calculated using a dose-response curve fit with nonlinear regression, and ordinary one-way ANOVA was used to compare difference between vaccine groups using Prism 9.3.1 (GraphPad Software, San Diego, CA, USA).

### Results and Discussion

### Selection of Genetic Distance Relevant to Cross-Immunoreactivity

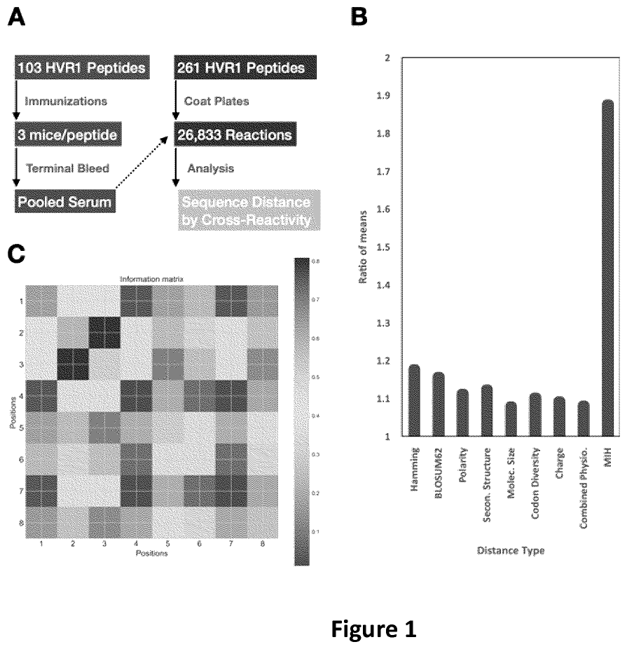

To identify HVR1 variants for immunization experiments, we modelled HVR1's genetic space, with the hypothesis that the space structure could inform variant selection and thus improve coverage. First, we explored how different measures of genetic distance were associated with a previously published cross-immunoreactivity dataset (20) of 26,883 pairwise reactions among 262 HVR1 variants (FIG. 1A). We compared the mean distance observed in pairs that did not cross-react, with the mean distance observed in pairs that did cross-react. If the ratio is 1, then the distance is not helping us to differentiate the two types of pairs, but the greater the ratio, the greater the relevance of the distance to cross-immunoreactivity. The ratio calculated using distances based on individual or joint physiochemical properties (21), Hamming distances (number of mismatches; ratio=1.19; t-test, p=1.3668E−279) or the BLOSUM62 scores (ratio=1.17; t-test, p=5.5753E−270) showed very similar results, all indicating low association with cross-immunoreactivity (FIG. 1B).

Considering importance of coordinated substitutions in HCV evolution (22), we devised a novel information-theory-based distance (Mahalanobis hamming) (23). The Mahalanobis hamming (MIH) distance considers the variability of each position (measured by entropy) and the existence of coordinated substitutions between position (measured by mutual information among positions pairs) (FIG. 1C). The mean of non-cross-reactive pairs was 1.89 times higher than the mean of cross-reactive pairs (t-test, p=8.88E−56), a ratio 58.7% greater than the second best, obtained with hamming distance. These results indicate that the MIH distance has a higher association with cross-immunoreactivity (FIG. 1B) and thus HVR1 variants with lower average MIH distance to other variants, are also more likely to be broadly cross-immunoreactive.

### HVR1 Sequence Space

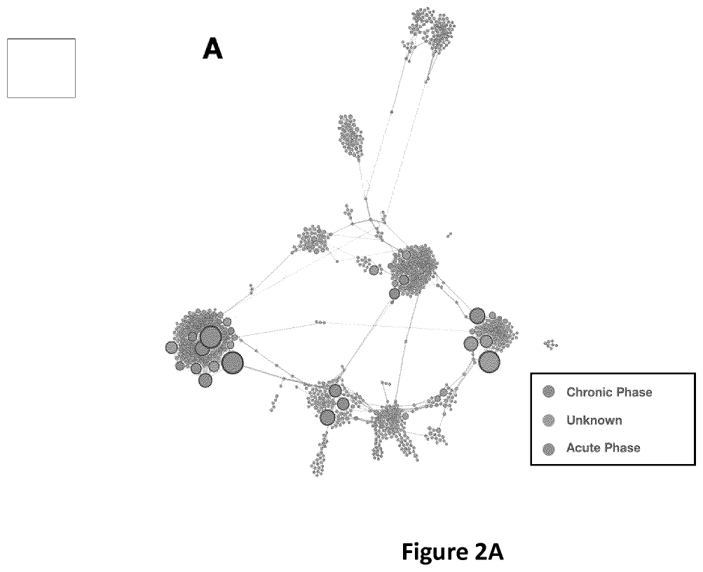

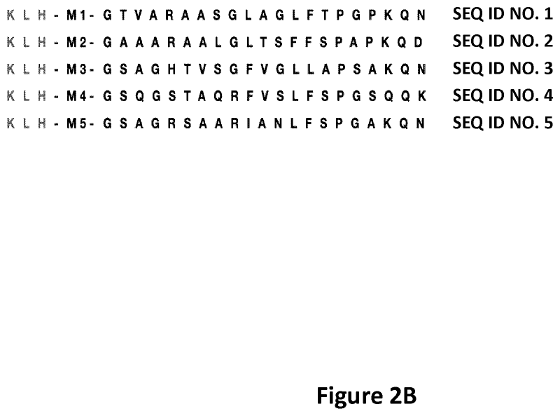

We then proceeded to measure the MIH distance among every pair of non-redundant (coding nonsynonymous) sequences in the extended global dataset of 12,245 HVR1 sequences. This matrix of distances was used to build a k-step network (FIG. 2A), which is equivalent to the union of all Minimum Spanning Trees and allows one to visualize the distances among all variants present. Thus, the network constitutes our model of the HVR1 sequence space, which we use to find modules and measure the centrality of each variant.

Given that early-acute phase variants (also referred to as Transmitted-Founder variants), are plausible targets for vaccine development, as they are the first variants encountered by the immune system (24), we studied their location in the HVR1 network. Mapping of HVR1 variants known to be collected during acute (n=119) and chronic (n=251) infection in the network showed that the acute HVR1 variants had a mean network centrality 9.73 times higher than the mean of chronic variants (t-test, p=0.0077), indicating their average MIH distance to other variants in the network is significantly reduced relative to chronic variants. This implies acute variants are more likely than chronic variants to be cross-reactive. In addition, these acute variants were not locally confined but were found globally distributed across the network and independent of HCV genotype. This indicates acute HVR1 variants, owing to their broad spread in the HVR1 genetic space, may possess complementary cross-immunoreactivities, which if combined, may provide broad cross reactivity leading to broad neutralization.

### Selection of HVR1 Variants for Immunization

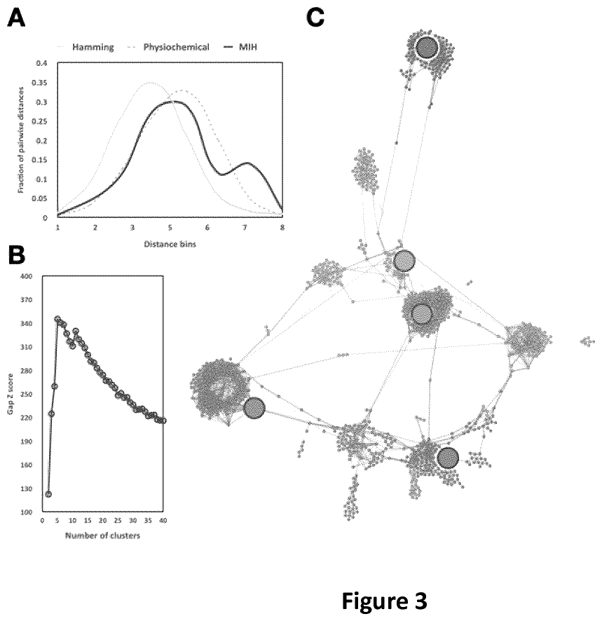

To discover the combination of variants most likely to possess complementary cross-immunoreactivities, we evaluated if the HVR1 network contained modules or clusters, with the hypothesis that each cluster would correspond to distinct HVR1 sub-phenotypes. The distribution of all pairwise MIH distances showed a bimodal distribution, suggesting the existence of modules (FIG. 3A). In contrast, distribution of the Hamming or physicochemical distances was unimodal, which indicates lack of a hierarchical structure in the HVR1 space modeled using these distances. The modular organization of the MIH-based network suggests that a combination of HVR1 variants selected from each module may be capable of inducing immune responses covering the entire space. Thus, we created modularity-maximizing partitions between 2 to 40 modules. We identified the five-module solution as the best one, given that it showed the highest difference between average within-module distances and the distance obtained by random partitioning of the same size (FIG. 3B). Finally, we identified the most central (acute-phase) variant in each of the five modules and selected them as immunogens for synthesis (FIG. 3C).

### Immunogens Elicit Cross-Reactive Antibodies

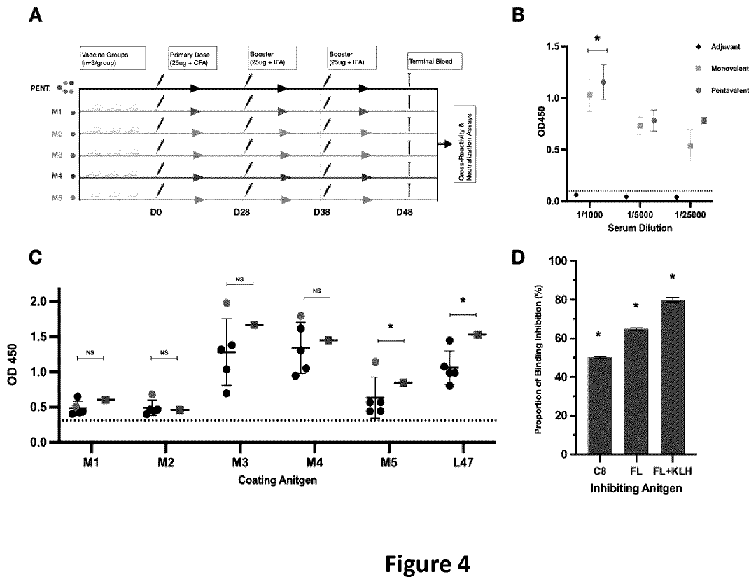

To evaluate if our candidate peptides were immunogenic, six groups of Balb/c mice (n=3 per group) were immunized with each of the peptides individually (monovalent) or combined (pentavalent), and terminally bled to characterize humoral responses (FIG. 4A). Both monovalent and pentavalent formulations elicited high-titer (1:25,000) peptide-specific antibodies following immunization, with higher reactivity observed with the pentavalent sera at the lowest dilution tested (t-test, p=0.003; FIG. 4B). Sera from mock immunized mice (adjuvant+PBS) were not reactive at any dilution tested (FIG. 4B). A concern in multivalent formulations is diminished reactivity to each of the individual, constituent immunogens. We therefore evaluated monovalent immunogenicity, based on self-reactivity, in comparison to the reactivity of the pentavalent immunized sera. Though we observed intrinsic differences in the antigenicity and immunogenicity of the monovalent immunogens, self-reactivity following pentavalent immunization was not inferior (FIG. 4C). Next, using competitive ELISA, we evaluated if antibodies elicited by pentavalent immunization targeted the C-terminal neutralizing epitope of HVR1. We observed significant binding inhibition when sera were pre-incubated with a peptide fragment comprising the C-terminal eight amino acids, suggesting antibodies elicited by the pentavalent formulation predominantly, though not exclusively, target the C-terminus of HVR1 (FIG. 4D).

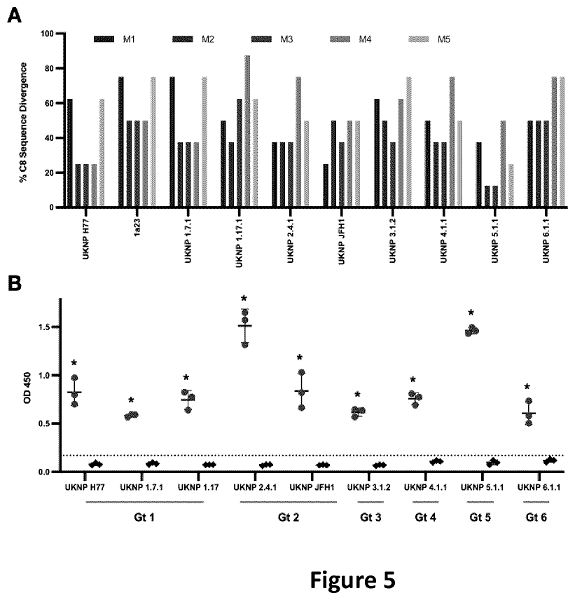

Next, we sought to characterize heterologous cross-reactivity using a panel of HVR1 peptides representing global genetic diversity. This was based on prior work to develop a standardized panel of HCV variants representing all major global genotypes, 1a intra-genotypic diversity, and the spectrum of neutralization resistance (25, 26). The sub-panel we selected was enriched for highly neutralization resistant variants maximally differing in genetic distance from our vaccine immunogens (50-87.5% sequence divergence) (FIG. 5A). By ELISA, we observed universal cross-reactivity of pentavalent sera with the panel of HVR1 peptides (FIG. 5B). No correlations between cross-reactivity and either HVR1 genotype or genetic distance to the pentavalent immunogens were observed (data not shown). These findings indicate that pentavalent immunization elicited broadly cross-reactive antibodies targeting the neutralizing epitope containing HVR1 C-terminus.

### Pentavalent Immunogen Elicits Broadly Neutralizing Antibodies

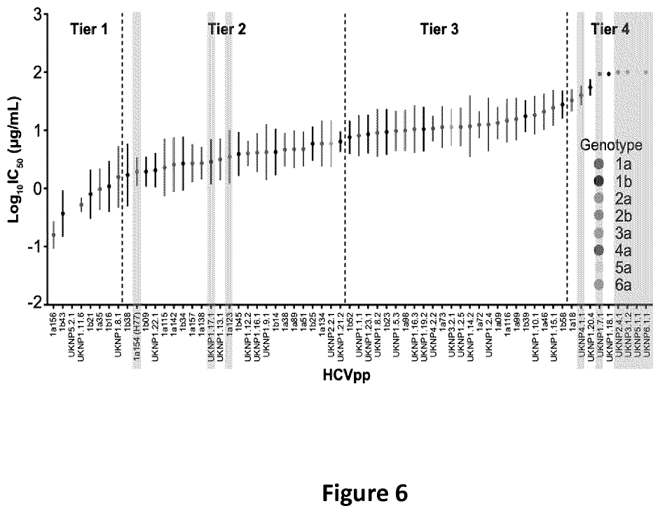

Our previous experiments demonstrated cross-reactivity to genetically diverse HVR1 peptides. Cross-reactivity is necessary but not sufficient for viral neutralization. We therefore sought to characterize the protective breadth of the antibodies elicited by pentavalent immunization using HCV pseudoparticles (HCVpp). Referring to FIG. 6, HCVpp neutralization sensitivity, ranked from most neutralization sensitive (Tier 1) to least neutralization sensitive (Tier 4). Highlighting denotes the HCVpp selected for neutralization assays

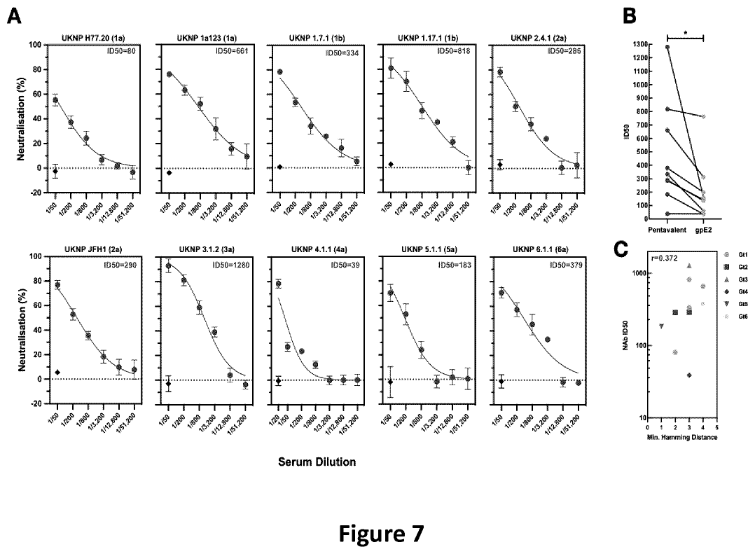

Briefly, for each HCV variant in our panel, HCVpp were generated, and residual infectivity in the presence of serial dilutions of mouse sera were used to calculate proportion neutralization and ID₅₀. We observed potent, universal neutralization across the HCVpp panel (FIG. 7A). Even highly neutralization resistant variants, such as UKNP3.1.2, which are almost completely resistant to neutralization by patient-derived sera (26), were potently neutralized by pentavalent sera (ID₅₀=1,280). Further, compared to a derivative of a gpE2 vaccine entering clinical trials, neutralization potency against UKNP3.1.2 was more than 10-fold higher, with average heterologous neutralization across the entire panel 2.32 fold higher (t-test, p=0.021; FIG. 7B). We found no relationship between sequence divergence from the pentavalent immunogens and neutralization resistance, with HCVpp UKNP1.17.1, which has the greatest Hamming distance from any immunogen in the formulation, potently neutralized (ID50=817; FIG. 7C). Collectively, these findings suggest the antibodies elicited by the pentavalent formulation can potently neutralize even extremely genetically distant variants, with no escape detected for any HCVpp in this antigenically diverse panel.

### Pentavalent Neutralization Breadth Exceeds Monovalent Constituents

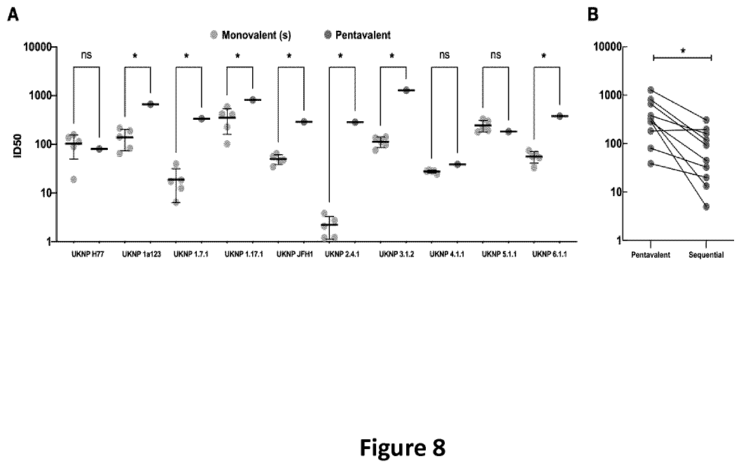

Next, we evaluated if pentavalent immunization elicited antibodies that could neutralize variants resistant to monovalent immunization. Interestingly, not only was pentavalent neutralization potency against the panel greater than average monovalent potency, but variants completely resistant to neutralization by every monovalent preparation were potently neutralized by pentavalent sera (UKNP1.7.1 and UKNP2.4.1). Across the panel, average pentavalent potency was 3.93-fold greater (t-test, p=0.009) than monovalent potency (ID₅₀=111), and for eight of the ten variants, was significantly greater than the most potent monovalent against each variant (FIG. 8A). We also compared the neutralization capacity of sera obtained following pentavalent immunization to sera obtained by sequentially immunizing mice with the same monovalent immunogens. Neutralization was not improved by sequentially administering the monovalent immunogens (mean ID₅₀=99), and was inferior to simultaneous (pentavalent) immunization (t-test, p=0.004; FIG. 8B), indicating that not only the valency, but also the method of immunization influences the humoral response. These findings suggest that a qualitatively distinct humoral response, rather than a summation of monovalent polyclonal responses, is operative in the broad neutralization observed following pentavalent immunization.

### Discussion

Vaccines are one of the most efficient public health tools to control infectious disease in human populations (27). However, development of vaccines to highly mutable viruses such as HIV, influenza virus, and HCV is greatly impeded by the genetic variability of dominant epitopes, immune responses against which are largely strain-specific, lacking the breadth of cross-immunoreactivity required for protection against a vast swarm of viral variants (28). HCV's HVR1 is a well-characterized example of a variable region eliciting only narrowly neutralizing antibodies following natural infection or vaccination (29). Here we present a new strategy, based on a novel model of the HVR1 genetic space, for designing complementary formulations of HVR1 antigens capable of directing the immune response to conserved epitopes within a sequence variable region. We show that immunization of mice with a mixture of HVR1 variants selected from each of the five genetic modules of the space produces antibodies demonstrating broad, potent, and superior neutralization activity.

This strategy is distinct from past vaccine approaches to variable viruses, which have attempted to direct immune responses to conserved epitopes (13, 14, 18). Though a rational approach to addressing antigenic variability, the limitations of conserved epitope targeting are evident in the natural history of HCV infection. Not only can conserved epitopes directly evolve to evade immune pressure, but diversifying selection on HVR1 persists even in the presence of conserved epitope targeting antibodies (6, 30, 31). This suggests that HVR1 can evolve to attenuate the neutralizing potency of not only HVR1-specific antibodies, but antibodies targeting other epitopes on the virion, which is mechanistically consistent with findings that HVR1 modulates the accessibility of conserved regions (32). That the pentavalent candidate reported here neutralized a panel of highly neutralization resistant, highly diverse HCV variants, suggests that a reappraisal of the role of variable epitopes in vaccine design is warranted, especially when their genetic space indicates the presence of functional constraints bounding variability.

Considering the proximity of HVR1 to the E2 receptor binding sites, the major function constraining the HVR1 genetic space is likely related to transmission and receptor binding. Indeed, HVR1 was shown to affect HCV infectivity by contributing to the optimal composition of virions and membrane fusion (15). In addition, it is a critical region for interaction between E2 and Scavenger Receptor class B type I (SR-BI) (33-35). Thus, if the HVR1 genetic space is largely shaped by balancing a single important function like transmissibility, with the diversifying selection of host immune pressure, there should be common structural features maintained by patterns of coordinated substitutions that permit immune evasion without compromising infectivity. Conservation of HVR1 size, physiochemical invariance, and extensive epistasis (ie coordinated substitutions) within HVR1 and between HVR1 and other positions in E2, support the existence of fitness-constrained structural features (22, 36). It is reasonable to expect that such conserved structural features, if properly presented to B-cells as antigenic epitopes, would elicit broadly neutralizing antibodies despite marked sequence divergence.

It is not clear what determines the differential presentation of these conserved epitopes among HVR1 variants. It is also unknown what determines cross-reactivity between any two HVR1 variants. Here we evaluated different measures of genetic distance to better understand both problems. We found that while simple sequence similarity (Hamming distance) could moderately discriminate between cross-reactive pairs, the novel MIH distance was markedly superior. This result is particularly important as it indicates that the distance captures the well-known fact that not all substitutions are equivalent (21), and that the more radical the substitution, measured by capacity to increase MIH distance, the more likely it will abrogate cross-immunoreactivity. When we explored the structure of the HVR1 sequence space using MIH, the network was found to be pentamodular, indicating that the structural features defining breadth of immunoreactivity, and mutual reactivity between any two variants, are distributed across 5 major HVR1 shapes. That acute-phase variants were also found to occupy positions of centrality within each module suggests that founder viruses can assume any of the 5 major shapes, and have a greater breadth of cross-immunoreactivity within each shape than chronic phase variants. This finding is in concert with the observation that early-acute phase variants, referred to as Transmitted-Founder variants, possess distinct, transmissibility enhancing phenotypes, and occupy central positions within the sequence space, affording greater mutational robustness from which to diversify once infection is established (37, 38). It is important that the acute HVR1 variants are not locally confined but are distributed across the k-step network, entirely independent of HCV genotype, as this indicates the existence of multiple Transmitted-Founder phenotypes, which must all be neutralized by a putative HCV vaccine.

The important observation is that these modules, or shapes, are convergent rather than defined by HCV genotypes and subtypes. Thus, a random selection of HVR1 variants from different genotypes may achieve, but does not guarantee, representation of all shapes. However, even the relatively immunodominant presentation of the conserved structural elements in high-centrality HVR1 variants may be affected by other amino acid sites, diverting the maturation of antibody producing B-cells in germinal centers towards a more strain- or module-specific recognition. This sub-dominance of the conserved epitope could be surmounted by the simultaneous presentation of the conserved epitope in different structural backgrounds to focus immune response on the common features rather than module-specific variations (39). This suggests that to achieve a universal broad neutralization, all potential shapes of the conserved epitope(s) may need to be simultaneously presented. Sequential exposure to each shape may instead successively direct maturation to module-specific features, limiting breadth of reactivity. This may explain why neutralization breadth and potency observed following sequential immunization with the five HVR1 peptides was inferior, and why chronic infection does not produce the breadth of neutralization observed following pentavalent immunization (30-31). The importance of simultaneous presentation is also supported by the finding that antibodies elicited by pentavalent immunization neutralized variants resistant to monovalent immunization. This synergistic interaction indicates that although the HVR1 variants selected for immunization were genetically distant, and occupied distinct modules, they shared the neutralizing epitope.

We recognize that a limited number of HVR1 variants were evaluated in the neutralization experiments. Although we selected known neutralization resistant and diverse HCVpp for the neutralization panel (25-26), the tested set is only an approximation of the entire HCV genetic space. However, the successful neutralization of all HCVpp clearly indicated the advantages of our approach. Our data demonstrates the synergistic effect for a mixture of five HVR1 variants. Whether this number can be reduced to identify a minimal number of variants to achieve a similar effect to reduce technological requirements for production of the potential vaccine requires further investigation. However, current prophylactic pneumococcal conjugate vaccines possess a valency of up to 20 (PCV20), demonstrating we are well within practical limits of vaccine technology. Our future studies will address these open questions and compare antibodies produced against individual HVR1 variants and the mixture of monovalent sera to understand the synergistic mechanism of pentavalent immunization for vaccine design. This will allow us to translate our in vitro neutralization data to real protection against HCV infection in vivo.

In conclusion, synergistic immune responses to HVR1 variants selected using a sequence space model accounting for the heterogeneity of each position and the interactions among amino acid positions, offer a novel approach to overcoming HCV genetic heterogeneity and the dominance of strain-specific immunity by directing the immune response to cross-immunoreactive neutralizing epitopes within HVR1. Application of this approach opens a new venue for the development of a universal HCV vaccine. This new approach may be generalizable to other highly mutable viruses.

Without being bound by any theory, there could be different models for how the polyvalent approach works.

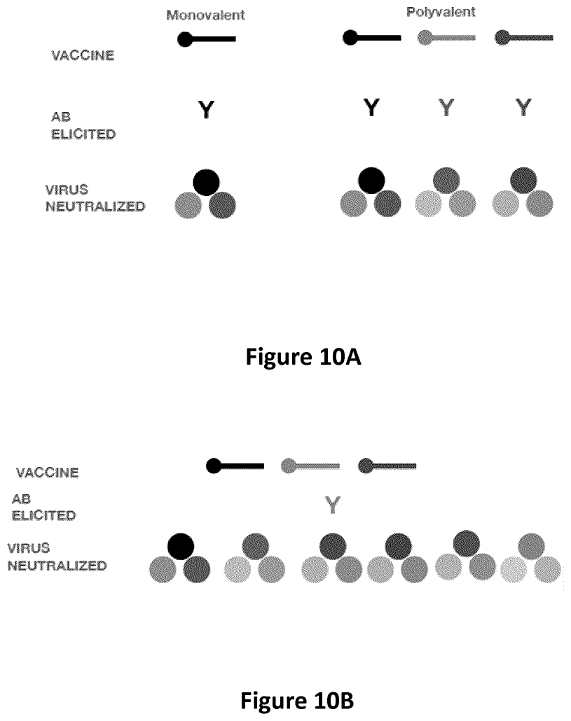

Referring to FIG. 10A, the polyvalent approach may work by eliciting Ab to each individual immunogen. These Ab can then neutralize viruses that are the same, or very closely related to the immunogen sequences. In this model, we would expect that the neutralization breadth of the polyvalent vaccine is equal to the summed neutralization breath of each monovalent vaccine (purely additive).

Referring to FIG. 10B, in a different model the present polyvalent approach works by eliciting a separate class of Ab that target physicochemically convergent features that are shared among all of the immunogens. We believe this results from greater stimulation of B-cell receptors that can recognize more than one immunogen in the polyvalent vaccine (e.g, if it can recognize all 5, it has 5× greater stimulation than a BCR that can only recognize 1 of the 5). In this model, we would expect the neutralization breadth of the polyvalent vaccine exceeds the summed neutralization breadth of the monovalent constituents. As shown in FIG. 7 discussed above, the current data support this model (only the pentavalent can neutralize the highly resistant virus 2.4.1).

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Perz, Joseph F., et al. ‘The Contributions of Hepatitis B Virus and Hepatitis C Virus Infections to Cirrhosis and Primary Liver Cancer Worldwide’. Journal of Hepatology, vol. 45, no. 4, October 2006, pp. 529-38. PubMed, https://doi.org/10.1016/j.jhep.2006.05.013
2. Dore, Gregory J., and Sahar Bajis. ‘Hepatitis C Virus Elimination: Laying the Foundation for Achieving 2030 Targets’. Nature Reviews Gastroenterology & Hepatology, vol. 18, no. 2, February 2021, pp. 91-92. www.nature.com, https://doi.org/10.1038/s41575-020-00392-3.
3. Page, Kimberly, et al. ‘Randomized Trial of a Vaccine Regimen to Prevent Chronic HCV Infection’. New England Journal of Medicine, vol. 384, no. 6, February 2021, pp. 541-49. Taylor and Francis+NEJM, https://doi.org/10.1056/NEJMoa2023345
4. Liang, T. Jake, et al. ‘Controlled Human Infection Model—Fast Track to HCV Vaccine?’ New England Journal of Medicine, vol. 385, no. 13, September 2021, pp. 1235-40. Taylor and Francis+NEJM, https://doi.org/10.1056/NEJMsb2109093
5. Pierce, Brian G., et al. ‘Viral Evasion and Challenges of Hepatitis C Virus Vaccine Development’. Current Opinion in Virology, vol. 20, October 2016, pp. 55-63. ScienceDirect, https://doi.org/10.1016/j.coviro.2016.09.004
6. von Hahn, Thomas, et al. ‘Hepatitis C Virus Continuously Escapes From Neutralizing Antibody and T-Cell Responses During Chronic Infection In Vivo’. Gastroenterology, vol. 132, no. 2, February 2007, pp. 667-78. ScienceDirect, https://doi.org/10.1053/j.gastro.2006.12.008
7. Martinez, Miguel Angel, and Sandra Franco. ‘Therapy Implications of Hepatitis C Virus Genetic Diversity’. Viruses, vol. 13, no. 1, January 2021, p. 41. www.mdpi.com, https://doi.org/10.3390/v13010041
8. Prentoe, Jannick, and Jens Bukh. ‘Hypervariable Region 1 in Envelope Protein 2 of Hepatitis C Virus: A Linchpin in Neutralizing Antibody Evasion and Viral Entry’. Frontiers in Immunology, vol. 9, 2018. Frontiers, https://www.frontiersin.org/article/10.3389/fimmu.2018.02146
9. Farci, Patrizia, et al. ‘Prevention of Hepatitis C Virus Infection in Chimpanzees by Hyperimmune Serum against the Hypervariable Region 1 of the Envelope 2 Protein’. Proceedings of the National Academy of Sciences, vol. 93, no. 26, December 1996, pp. 15394-99. DOI.org (Crossref), https://doi.org/10.1073/pnas.93.26.15394
10. Shimizu, Yohko K., et al. ‘A Hyperimmune Serum against a Synthetic Peptide Corresponding to the Hypervariable Region 1 of Hepatitis C Virus Can Prevent Viral Infection in Cell Cultures’. Virology, vol. 223, no. 2, September 1996, pp. 409-12. Science Direct, https://doi.org/10.1006/viro. 1996.0497
11. Bartosch, Birke, et al. ‘In Vitro Assay for Neutralizing Antibody to Hepatitis C Virus: Evidence for Broadly Conserved Neutralization Epitopes’. Proceedings of the National Academy of Sciences, vol. 100, no. 24, November 2003, pp. 14199-204. DOI.org (Crossref), https://doi.org/10.1073/pnas.2335981100
12. Drummer, Heidi E. ‘Challenges to the Development of Vaccines to Hepatitis C Virus That Elicit Neutralizing Antibodies’. Frontiers in Microbiology, vol. 5, 2014. Frontiers, https://www.frontiersin.org/article/10.3389/fmicb.2014.00329
13. Pierce, Brian G., et al. ‘Structure-Based Design of Hepatitis C Virus Vaccines That Elicit Neutralizing Antibody Responses to a Conserved Epitope’. Journal of Virology, vol. 91, no. 20, October 2017, pp. e01032-17. PubMed, https://doi.org/10.1128/JVI.01032-17
14. Tzarum, Netanel, et al. ‘An Alternate Conformation of HCV E2 Neutralizing Face as an Additional Vaccine Target’. Science Advances, vol. 6, no. 30, July 2020, p. eabb5642. PubMed Central, https://doi.org/10.1126/sciadv.abb5642
15. Bankwitz, Dorothea, et al. ‘Hepatitis C Virus Hypervariable Region 1 Modulates Receptor Interactions, Conceals the CD81 Binding Site, and Protects Conserved Neutralizing Epitopes’. Journal of Virology, vol. 84, no. 11, June 2010, pp. 5751-63. PubMed, https://doi.org/10.1128/JVI.02200-09
16. Prentoe, Jannick, et al. ‘Hypervariable Region 1 Shielding of Hepatitis C Virus Is a Main Contributor to Genotypic Differences in Neutralization Sensitivity’. Hepatology (Baltimore, Md.), vol. 64, no. 6, December 2016, pp. 1881-92. PubMed Central, https://doi.org/10.1002/hep.28705
17. Prentoe, Jannick, et al. ‘Hypervariable Region 1 and N-Linked Glycans of Hepatitis C Regulate Virion Neutralization by Modulating Envelope Conformations’. Proceedings of the National Academy of Sciences, vol. 116, no. 20, May 2019, pp. 10039-47. DOI.org (Crossref), https://doi.org/10.1073/pnas.1822002116.
18. Law, John L. M., et al. ‘Role of the E2 Hypervariable Region (HVR1) in the Immunogenicity of a Recombinant Hepatitis C Virus Vaccine’. Journal of Virology, vol. 92, no. 11, June 2018, pp. e02141-17. PubMed, https://doi.org/10.1128/JVI.02141-17.
19. Law, Mansun, et al. ‘Broadly Neutralizing Antibodies Protect against Hepatitis C Virus Quasispecies Challenge’. Nature Medicine, vol. 14, no. 1, January 2008, pp. 25-27. www.nature.com, https://doi.org/10.1038/nm1698
20. Campo, David S., et al. ‘Hepatitis C Virus Antigenic Convergence’. Scientific Reports, vol. 2, no. 1, February 2012, p. 267. www.nature.com, https://doi.org/10.1038/srep00267
21. Atchley, William R., et al. ‘Solving the Protein Sequence Metric Problem’. Proceedings of the National Academy of Sciences, vol. 102, no. 18, May 2005, pp. 6395-400. DOI.org (Crossref), https://doi.org/10.1073/pnas.0408677102
22. Campo, D. S., et al. ‘Coordinated Evolution of the Hepatitis C Virus’. Proceedings of the National Academy of Sciences, vol. 105, no. 28, July 2008, pp. 9685-90. DOI.org (Crossref), https://doi.org/10.1073/pnas.0801774105
23. Campo, D. Mosa, A. Khudyakov, Y. Oral presentation. “Entropy-based distance for categorical data: Application to the clustering of protein sequences”. 10th Workshop on Computational Advances in Molecular Epidemiology (CAME2021) at the 11th International Conference on Computational Advances in Bio and Medical Sciences (ICCABS). Dec. 16-18, 2021. Virtual conference.
24. Li, Hui, et al. ‘Molecular Identification of Transmitted/Founder Hepatitis C Viruses and Their Progeny by Single Genome Sequencing’. Methods in Molecular Biology (Clifton, N.J.), vol. 1911, 2019, pp. 139-55. PubMed, https://doi.org/10.1007/978-1-4939-8976-8_9
25. Urbanowicz, Richard A., et al. ‘A Diverse Panel of Hepatitis C Virus Glycoproteins for Use in Vaccine Research Reveals Extremes of Monoclonal Antibody Neutralization Resistance’. Journal of Virology, vol. 90, no. 7, December 2015, pp. 3288-301. PubMed, https://doi.org/10.1128/JVI.02700-15
26. Salas, Jordan H., et al. ‘An Antigenically Diverse, Representative Panel of Envelope Glycoproteins for Hepatitis C Virus Vaccine Development’. Gastroenterology, vol. 162, no. 2, February 2022, pp. 562-74. ScienceDirect, https://doi.org/10.1053/j.gastro.2021.10.005
27. Pollard, Andrew J., and Else M. Bijker. ‘A Guide to Vaccinology: From Basic Principles to New Developments’. Nature Reviews Immunology, vol. 21, no. 2, February 2021, pp. 83-100. www.nature.com, https://doi.org/10.1038/s41577-020-00479-7
28. Kennedy, Richard B., et al. ‘Current Challenges in Vaccinology’. Frontiers in Immunology, vol. 11, 2020. Frontiers, https://www.frontiersin.org/article/10.3389/fimmu.2020.01181
29. Vieyres, Gabrielle, et al. ‘Characterization of Antibody-Mediated Neutralization Directed against the Hypervariable Region 1 of Hepatitis C Virus E2 Glycoprotein’. The Journal of General Virology, vol. 92, no. Pt 3, March 2011, pp. 494-506. PubMed Central, https://doi.org/10.1099/vir.0.028092-0
30. Duan, Hongying, et al. ‘Hepatitis C Virus with a Naturally Occurring Single Amino-Acid Substitution in the E2 Envelope Protein Escapes Neutralization by Naturally-Induced and Vaccine-Induced Antibodies’. Vaccine, vol. 28, no. 25, June 2010, pp. 4138-44. ScienceDirect, https://doi.org/10.1016/j.vaccine.2010.04.024.
31. Bailey, Justin R., et al. ‘Constraints on Viral Evolution during Chronic Hepatitis C Virus Infection Arising from a Common-Source Exposure’. Journal of Virology, vol. 86, no. 23, December 2012, pp. 12582-90. PubMed Central, https://doi.org/10.1128/JVI.01440-12.
32. Stejskal, Lenka, et al. ‘Flexibility and Intrinsic Disorder Are Conserved Features of Hepatitis C Virus E2 Glycoprotein’. PLOS Computational Biology, vol. 16, no. 2, February 2020, p. e1007710. PubMed Central, https://doi.org/10.1371/journal.pcbi.1007710.
33. Scarselli, Elisa, et al. ‘The Human Scavenger Receptor Class B Type I Is a Novel Candidate Receptor for the Hepatitis C Virus’. The EMBO Journal, vol. 21, no. 19, October 2002, pp. 5017-25. PubMed, https://doi.org/10.1093/emboj/cdf529.
34. Bartosch, Birke, et al. ‘An Interplay between Hypervariable Region 1 of the Hepatitis C Virus E2 Glycoprotein, the Scavenger Receptor BI, and High-Density Lipoprotein Promotes Both Enhancement of Infection and Protection against Neutralizing Antibodies’. Journal of Virology, vol. 79, no. 13, July 2005, pp. 8217-29. DOI.org (Crossref), https://doi.org/10.1128/JVI.79.13.8217-8229.2005
35. Johnson, Janelle, et al. ‘A Recombinant Hepatitis C Virus Genotype 1a E1/E2 Envelope Glycoprotein Vaccine Elicits Antibodies That Differentially Neutralize Closely Related 2a Strains through Interactions of the N-Terminal Hypervariable Region 1 of E2 with Scavenger Receptor B1’. Journal of Virology, vol. 93, no. 22, October 2019, pp. e00810-19. PubMed Central, https://doi.org/10.1128/JVI.00810-19
36. Penin, François, et al. ‘Conservation of the Conformation and Positive Charges of Hepatitis C Virus E2 Envelope Glycoprotein Hypervariable Region 1 Points to a Role in Cell Attachment’. Journal of Virology, vol. 75, no. 12, June 2001, pp. 5703-10. DOI.org (Crossref), https://doi.org/10.1128/JVI.75.12.5703-5710.2001
37. Campo, David S., et al. ‘Transmissibility of Intra-Host Hepatitis C Virus Variants’. BMC Genomics, vol. 18, no. Suppl 10, December 2017, p. 881. PubMed Central, https://doi.org/10.1186/s12864-017-4267-4.
38. Astrakhantseva, I. V., et al. ‘Variation in Physicochemical Properties of the Hypervariable Region 1 during Acute and Chronic Stages of Hepatitis C Virus Infection’. 2011 IEEE International Conference on Bioinformatics and Biomedicine Workshops (BIBMW), 2011, pp. 72-78. IEEE Xplore, https://doi.org/10.1109/BIBMW.2011.6112357.
39. Dutta, Sheetij, et al. ‘Overcoming Antigenic Diversity by Enhancing the Immunogenicity of Conserved Epitopes on the Malaria Vaccine Candidate Apical Membrane Antigen-1’. PLOS Pathogens, vol. 9, no. 12, 2013, p. e1003840. PubMed, https://doi.org/10.1371/journal.ppat. 1003840
40. Pickett, Brett E., et al. ‘Virus Pathogen Database and Analysis Resource (ViPR): A Comprehensive Bioinformatics Database and Analysis Resource for the Coronavirus Research Community’. Viruses, vol. 4, no. 11, November 2012, pp. 3209-26. PubMed, https://doi.org/10.3390/v4113209
41. Icer Baykal, Pelin B., et al. ‘Quantitative Differences between Intra-Host HCV Populations from Persons with Recently Established and Persistent Infections’. Virus Evolution, vol. 7, no. 1, December 2020, p. veaa103. PubMed Central, https://doi.org/10.1093/ve/veaa103.
42. Quirin, Arnaud, et al. ‘A Quick MST-Based Algorithm to Obtain Pathfinder Networks (∞, n−1)’. Journal of the American Society for Information Science and Technology, vol. 59, no. 12, October 2008, pp. 1912-24. DOI.org (Crossref), https://doi.org/10.1002/asi.20904
43. Campo, David S., et al. ‘Next-Generation Sequencing Reveals Large Connected Networks of Intra-Host HCV Variants’. BMC Genomics, vol. 15, no. 5, July 2014, p. S4. BioMed Central, https://doi.org/10.1186/1471-2164-15-S5-S4
44. Campo, D S, et al. ‘Accurate Genetic Detection of Hepatitis C Virus Transmissions in Outbreak Settings’. The Journal of Infectious Diseases, vol. 213, no. 6, March 2016, pp. 957-65. PubMed Central, https://doi.org/10.1093/infdis/jiv542
45. Bastian, Mathieu, et al. ‘Gephi: An Open Source Software for Exploring and Manipulating Networks’. Proceedings of the International AAAI Conference on Web and Social Media, vol. 3, no. 1, March 2009, pp. 361-62. ojs.aaai.org, https://ojs.aaai.org/index.php/ICWSM/article/view/13937
46. Tibshirani, Robert, et al. ‘Estimating the Number of Clusters in a Data Set via the Gap Statistic’. Journal of the Royal Statistical Society: Series B (Statistical Methodology), vol. 63, no. 2, 2001, pp. 411-23. DOI.org (Crossref), https://doi.org/10.1111/1467-9868.00293.
47. Mosa, Alexander I., et al. ‘A Bivalent HCV Peptide Vaccine Elicits Pan-Genotypic Neutralizing Antibodies in Mice’. Vaccine, vol. 38, no. 44, October 2020, pp. 6864-67. PubMed, https://doi.org/10.1016/j.vaccine.2020.08.066.
48. Mosa, Alexander I., et al. ‘Role of HVR1 Sequence Similarity in the Cross-Genotypic Neutralization of HCV’. Virology Journal, vol. 17, September 2020, p. 140. PubMed Central, https://doi.org/10.1186/s12985-020-01408-9.
49. Yu, Xuesong, et al. ‘Statistical Approaches to Analyzing HIV-1 Neutralizing Antibody Assay Data’. Statistics in Biopharmaceutical Research, vol. 4, no. 1, January 2012, pp. 1-13. PubMed Central, https://doi.org/10.1080/19466315.2011.633860

1. A peptide comprising the sequence set forth in any one of SEQ ID Nos. 1-5.

2. The peptide of claim 1, consisting of the sequence set forth in any one of SEQ ID Nos. 1-5.

3. The peptide of claim 1, conjugated to a vaccine-suitable carrier protein.

4. The peptide of claim 1, wherein the carrier protein is N-terminally conjugated.

5. The peptide of claim 1, wherein the carrier protein is C-terminally conjugated.

6. The peptide of claim 1, wherein the carrier protein is keyhole limpet hemocyanin (KLH).

7. The peptide of claim 1, wherein the peptide is conjugated to KLH via a suitable linker, preferably a maleimide linkage.

8. A nucleic acid encoding the peptide of claim 1.

9. A vector comprising the nucleic acid of claim 8.

10. A vaccine composition comprising the peptide of claim 1 or the nucleic acids encoding them, along with a pharmaceutically acceptable carrier.

11. A vaccine composition comprising the peptide of claim 1 or the nucleic acids encoding them, and an adjuvant.

12. The vaccine composition of claim 10, comprising at least two different peptides of claim 1, or the nucleic acids encoding them, wherein the at least two different peptides comprise two of SEQ ID Nos. 1-5.

13. The vaccine composition of claim 12, comprising at least three different peptides, or the nucleic acids encoding them, wherein the at least three different peptides comprise three of SEQ ID Nos. 1-5.

14. The vaccine composition of claim 12, comprising at least four different peptides, or the nucleic acids encoding them, wherein the at least four different peptides comprise four of SEQ ID Nos. 1-5.

15. The vaccine composition of claim 12, comprising at least five different peptides, or the nucleic acids encoding them, wherein the at least five different peptides comprise all five of SEQ ID Nos. 1-5.

16. (canceled)

17. (canceled)

18. A method of immunizing a subject against HCV infection comprising administrating to the subject, the vaccine composition of claim 10.

19. A method for producing a multivalent vaccine comprising a plurality of peptides or the nucleic acids encoding them, the method comprising:
a) selecting a target epitope;
b) mapping a sequence space for the targeted epitope;
c) synthesizing peptides covering the sequence space;
d) immunizing animals with the peptides;
e) evaluating cross-reactivity between animal sera to determine a predictive feature of reactivity;
f) creating a network of haplotypes wherein distance between nodes is based on the predictive feature;
g) creating clusters of haplotypes using a mathematical model;
h) selecting a representative haplotype from each cluster for the plurality of peptides, or the nucleic acids encoding them, in the multivalent vaccine.

20. The method of claim 19, wherein the predictive feature is sequence similarity, physicochemical, or Mahalanobis Hamming Distance (MIH).

21-27. (canceled)


SEQUENCE LISTING (5 sequences)

SEQ ID NO: 1 - protein, 21 aa, synthetic construct
GTVARAASGLAGLFTPGPKQN

SEQ ID NO: 2 - protein, 21 aa, synthetic construct
GAAARAALGLTSFFSPAPKQD

SEQ ID NO: 3 - protein, 21 aa, synthetic construct
GSAGHTVSGFVGLLAPSAKQN

SEQ ID NO: 4 - protein, 21 aa, synthetic construct
GSQGSTAQRFVSLFSPGSQQK

SEQ ID NO: 5 - protein, 21 aa, synthetic construct
GSAGRSAARIANLFSPGAKQN